(19) United States
(12) Patent Application Publication
(10) Pub. No.: US 2010/0286688 A1
(43) Pub. Date: Nov. 11, 2010

(54) FLEXIBLE ABLATION CLAMP

(76) Inventors: James David Hughett, SR., Liberty Township, OH (US); Salvatore Privitera, Mason, OH (US); Keith Edward Martin, Mason, OH (US); Kenneth Lance Miller, Hamilton, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 12/775,777

(22) Filed: May 7, 2010

Related U.S. Application Data
(60) Provisional application No. 61/176,544, filed on May 8, 2009.

(51) Int. Cl. A61B 18/18; A61B 18/14

(52) U.S. Cl. 606/33; 606/41

(57) ABSTRACT

Ablation devices comprising elongated flexible members that are repositioned to at least partially surround tissue to be ablated. The exemplary ablation instruments utilize rigid or semi-rigid devices that are either affixed to the flexible member or repositionable along the flexible member. The clamping and ablating surfaces include electrodes or other means for delivering ablation energy. The electrodes may be brought into proximity with each other so as to provide a uniform force profile across the tissue to be ablated. In such a circumstance, once the tissue between the clamping and ablating surfaces is compressed, the electrodes are activated to create an ablation lesion.

### CROSS REFERENCE TO RELATED APPLICATIONS

The present application claims the benefit of U.S. Provisional Patent Application Ser. No. 61/176,544, entitled, “FLEXIBLE ABLATION CLAMP,” filed May 8, 2009, the disclosure of which is incorporated herein by reference.

### RELATED ART

1. Field of the Invention

The present disclosure is directed to an apparatus and method for ablating tissue, such as during a minimally invasive procedure.

2. Brief Discussion of Related Art

Atrial fibrillation is the most common cardiac arrhythmia and involves the upper two chambers or atria of the heart. In atrial fibrillation, the normal electrical impulses that are generated by the sinoatrial node are overwhelmed by disorganized electrical impulses that originate in the atria and pulmonary veins, leading to conduction of irregular impulses to the ventricles that generate the heart beat.

One method of treating atrial fibrillation is to create a continuous, transmural line of scar tissue, (i.e., a lesion) that encircles the pulmonary veins to electrically isolate the pulmonary veins from the rest of the atria. See, e.g., U.S. Pat. No. 6,517,536, which is incorporated herein by reference.

### INTRODUCTION TO THE INVENTION

The devices disclosed herein comprise an elongated flexible member that may be introduced into the body in a minimally invasive manner, such as through a small (approximately 8 mm) introducer or trocar, and maneuvered around the tissue to be ablated and back toward the user, so that both ends of the flexible member extend outside of the body. Ablation and clamping surfaces are provided by rigid or semi-rigid members that are either affixed to the flexible member, or introduced over the ends of the flexible member, or through a conduit within the flexible member. The clamping and ablating surfaces include electrodes or other means for delivering ablation energy. The electrodes may be brought into proximity with each other by a cinching member associated with the ends of the flexible member. In such a circumstance, once the cinching member is used to compress the tissue between the clamping and ablating surfaces, the electrodes are activated to create an ablation.

### BRIEF DESCRIPTION OF THE DRAWINGS

FIG. 1 is a profile view of a first exemplary embodiment of a flexible ablation clamp according to the present disclosure.

FIG. 2 is an elevated perspective view of a second exemplary embodiment of a flexible ablation clamp according to the present disclosure.

FIG. 3 is an elevated perspective view of a third exemplary embodiment of a flexible ablation clamp according to the present disclosure.

FIG. 4 is an elevated perspective view of a fourth exemplary embodiment of a flexible ablation clamp according to the present disclosure.

FIG. 5 is an elevated perspective view of a fifth exemplary embodiment of a flexible ablation clamp according to the present disclosure.

FIG. 6 is an elevated perspective view of a distal portion of the fourth exemplary embodiment of FIG. 5.

FIG. 7 is another elevated perspective view of a distal portion of the fourth exemplary embodiment of FIG. 5.

FIG. 8 is an elevated perspective view of the distal portion of the fourth exemplary embodiment of FIG. 5 in a linear configuration

FIG. 9 are exemplary pressure diagrams showing nonuniform and uniform force profiles with respect to tissue to be ablated.

FIG. 10 is an elevated perspective view of a sixth exemplary embodiment of a flexible ablation clamp according to the present disclosure.

FIG. 11 is an elevated perspective view of the sixth exemplary embodiment of FIG. 10 with the sections coupled and folded over one another.

FIG. 12 is another elevated perspective view of the sixth exemplary embodiment of FIG. 10 with the sections coupled and folded over one another.

FIG. 13 is a profile view of the sixth exemplary embodiment of FIG. 10 with the sections coupled and folded over one another.

FIG. 14 is an elevated perspective view of the sixth exemplary embodiment of FIG. 10 with the sections uncoupled and no longer folded over one another.

FIG. 15 is an elevated perspective view of the sixth exemplary embodiment of FIG. 10 with the sections uncoupled and no longer folded over one another, while the bladders are deflated.

FIG. 16 is an overhead view of a seventh exemplary embodiment of a flexible ablation clamp according to the present disclosure.

FIG. 17 is an overhead view of the flexible member of the seventh exemplary embodiment of FIG. 16.

FIG. 18 is an overhead view of the filament, ablation electrodes, and transmission line of the seventh exemplary embodiment of FIG. 16.

### DETAILED DESCRIPTION

The exemplary embodiments of the present disclosure are described and illustrated below to encompass an apparatus and method for ablating tissue such as, without limitation, tissue on the surface of the heart via a minimally invasive procedure. Of course, it will be apparent to those of ordinary skill in the art that the embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

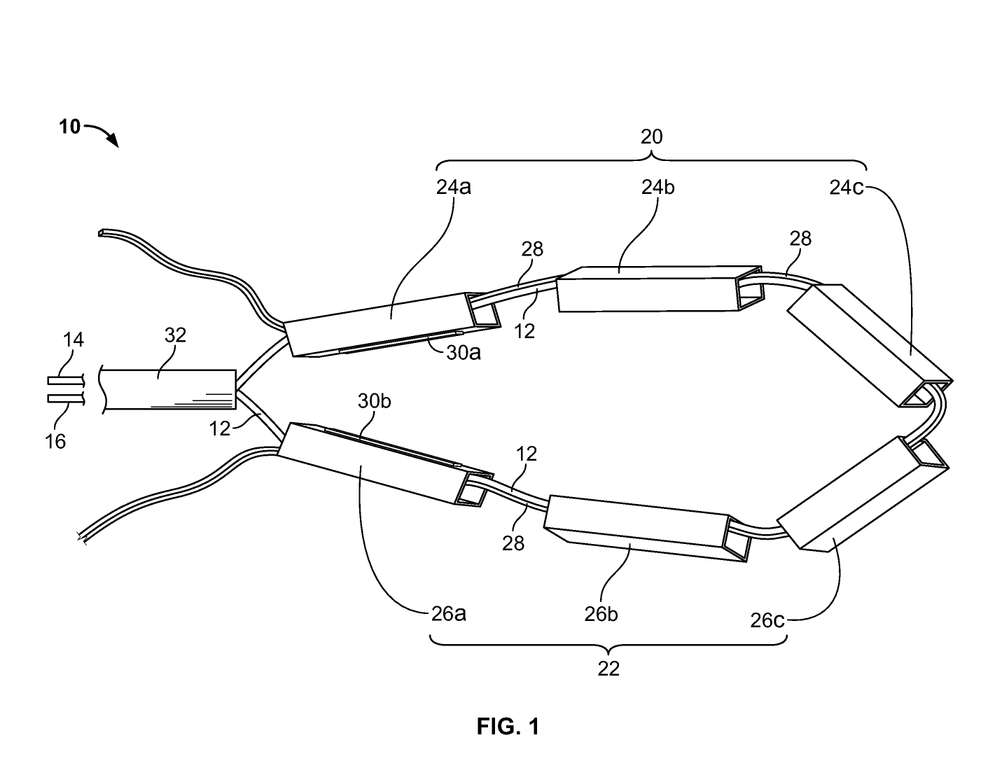

With reference to FIG. 1, a first exemplary embodiment of a flexible ablation clamp 10 comprises an elongated flexible member 12. In exemplary form, the elongated flexible member 12 includes a length sufficient for it to be capable of being introduced into the body of a patient through a sub-xyphoid access port, and then encircling the tissue in question, while the ends 14, 16 of the flexible member 12 are disposed outside of the body. By way of example, this tissue in question may comprise the pulmonary veins (both inferior and superior) associated with a mammalian heart.

The elongated flexible member 12 comprises a strip or ribbon that is formed, at least in part, of an elastomer, such as a polyethylene or polyurethane. One such material suitable for this use is Pellethane polyurethane, Series 2363, available from The Dow Chemical Company (www.dow.com). Various geometries may be employed for the elongated member 12, so no single geometry or cross-section is required. With that said, it may be advantageous for the geometry or cross-section to allow both ends 14, 16 of the elongated flexible member to freely pass through the introducer/trocar in a minimally invasive procedure. But, by way of example, the elongated flexible member 12 has a length from about 24 to 36 inches.

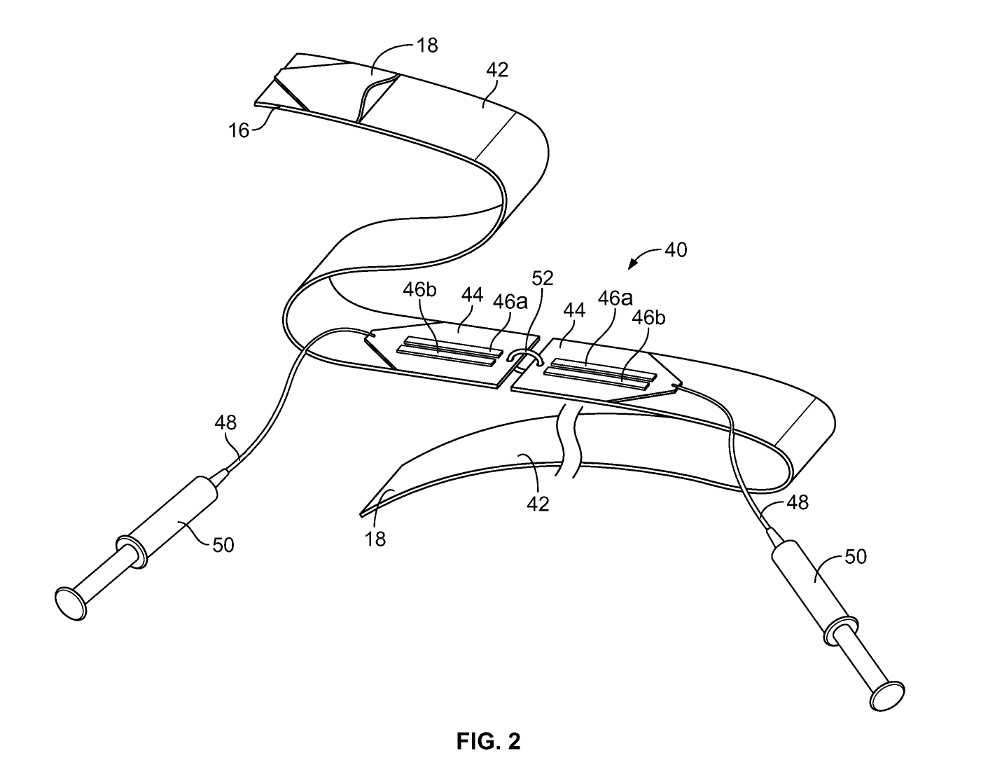

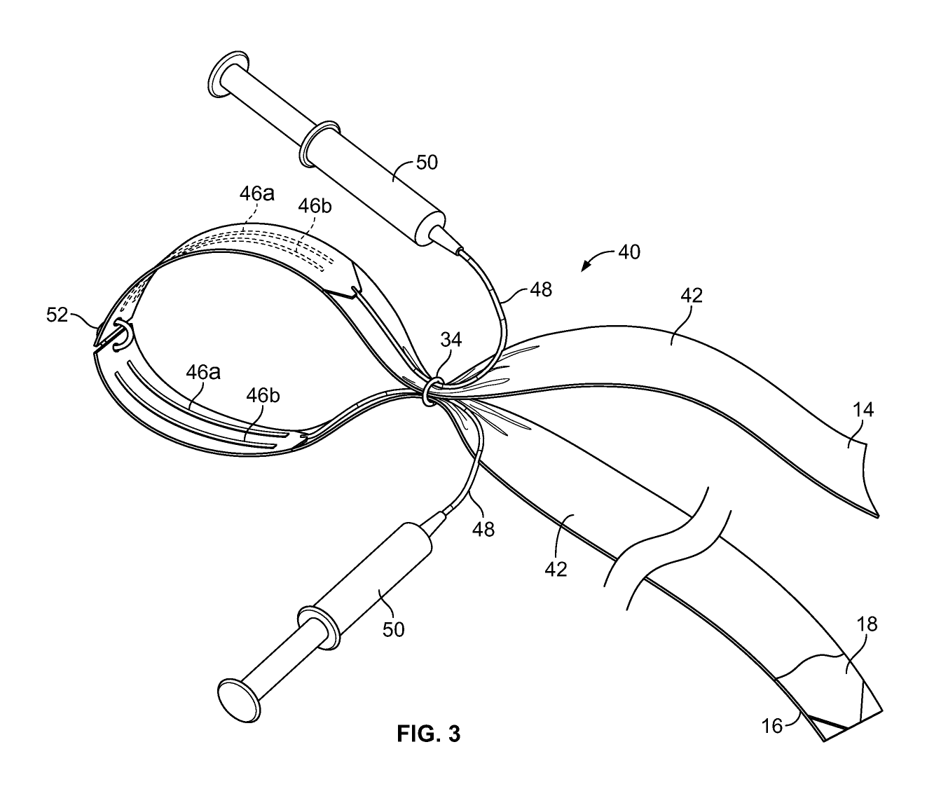

The flexible member 12 may also include pockets formed on or adjacent one or both of its ends 14, 16 (see, e.g., pocket 18 in FIGS. 2 and 3) that are adapted to cooperate with a positioning tool or instrument, such as the LumiTip dissector available from AtriCure, Inc., West Chester, Ohio (www.atricure.com), for guiding the flexible member around the tissue to be ablated. In this regard, see, e.g., U.S. Patent Application Publication No. 2006/0167478, the disclosure of which is incorporated herein by reference.

First and second sets 20, 22 of elongated, generally rigid rectangular tubular sleeve members 24a, 24b, 24c, 26a, 26h, 26c are received over the elongated flexible member 12. As shown in FIG. 1, the first set 20 comprises three separate sleeve members 24a, 24b, 24c, and the second set 22 also comprises three separate sleeves. It should be noted, however, that more or less than three sleeves are also within the scope of the invention. The sleeve members 24a-c, 26a-c may embody various sizes, but in exemplary form include lengths ranging between about 10 mm and 70 mm. For example, if an ablation lesion needs to have a length of approximately 50 mm to 70 mm, the cumulative length of the sleeve members 24a-c, 26a-c in each set 20, 22 that are used to create the ablation lesion would be from about 50 mm to 70 mm to achieve such a result.

It should also be noted that one or more of the sleeve members 24a-c, 26a-c may not clamp tissue therebetween. For example, in a circumstance where all six sleeve members 24a-c, 26a-c are utilized, but only the first pair of sleeve members 24a, 26a includes tissue therebetween to be ablated, the respective lengths of the sleeve members could be adjusted in order to create an ablation lesion of the desired length.

While each illustrated sleeve member 24a-c, 26a-c has a generally rectangular cross-section that remains constant along its length, other cross-sectional shapes may be used such as, without limitation, elliptical, triangular, hexagonal, and semi-circular.

The sleeve members 24a-c, 26a-c may be fabricated from any surgically suitable material that preferably has a relatively moderate to high dielectric value. Exemplary materials suitable for forming the sleeve members include, without limitation, thermoplastic resins (e.g., acrylonitride butadiene styrene, polyetherlmide).

The sleeve members 24a-c, 26a-c in each set may be interconnected by an elastomeric member or material 28. Optionally, the first set 20 of sleeve members 24a-c may be connected by an elastomeric member or material to the second set 22 of sleeve members 26a-c. As illustrated, the first set of sleeve members 20 is not interconnected to the second set of sleeve members 22, thus permitting each set 20, 22 to be introduced individually over respective opposite ends 14, 16 of the elongated flexible member 12 and advanced into position.

An ablation energy delivery member, such as an RF electrode 30, is associated with at least one sleeve member 24a-c, 26a-c in each set 20, 22. As shown in FIG. 1, sleeve members 24a and 26a are provided with an elongated RF electrode 30a, 30b located on a generally flat, exposed surface of the sleeve member. However, any or all of the sleeve members 24a-c, 26a-c in each set 20, 22 may be provided with at least one electrode 30, depending on the ablation pattern desired. In exemplary form, opposing sleeve members 24a, 26a are provided with a pair of RF electrodes 30a, 30b. The electrodes 30 are connected to an RF energy source (not shown) so that opposing electrodes 24a, 26a, for example, have the opposite polarity.

After the sleeve members 24a-c, 26a-c are positioned on the elongated flexible member 12 and positioned proximate the tissue to be ablated (e.g., flexible member 12 loops around the tissue to be ablated), the ends 14, 16 of the elongated flexible member 12 are drawn taut and cinched down so as to cause the sleeve members to compress the tissue to be ablated therebetween. As shown in FIG. 1, an elongated tubular member 32 is provided for cinching down the flexible member 12, with the ends 14, 16 of the flexible member 12 being threaded through the tubular member so that the ends may be grasped and pulled while advancing the tubular member around the flexible member. As soon as the elongated flexible member is cinched down, the electrodes may be activated to deliver ablation energy to the atrium for forming the line of ablation.

Turning to FIGS. 2 and 3, an alternative embodiment of a flexible ablation clamp 40 comprises an elongated flexible member 42 similar to that in FIG. 1 in terms of its general characteristics. However, instead of sets 20, 22 of elongated sleeve members 24a-c, 26a-c that carry the ablation electrodes 30a, 30b, the device 40 supports at least one pair of elongated inflatable members 44 that are spaced apart on opposite portions the elongated flexible member 42. Each inflatable member 44 may include a semi-rigid elongated member or beam (not shown) associated therewith. Each of the inflatable members 44 includes a pair of elongated electrodes 46a, 46b associated with corresponding surfaces so what when the flexible member 42 is looped around the tissue to be ablated, the elongated electrodes are oriented to face one another. An RF power source (not shown) is in communication with the electrodes 46a, 46b to provide RF energy.

Once the elongated member 40 is positioned about the tissue to be ablated, the inflatable members 44 are inflated, to provide sufficient rigidity so that when the elongated flexible member 40 is cinched down, the inflatable members 44 will compress the tissue to be ablated therebetween, proximate the electrodes 46a, 46b of the two inflatable members. Prior to inflation, the inflatable members 44 are generally compliant, but upon inflation, the members 44 are non-compliant as a result of the desired amount of rigidity.

The inflatable members 44 may be inflated using a gas, liquid, or mixture of the two using a pump for delivering pressurized fluid through an elongated inflation lumen 48. As shown in FIGS. 2 and 3, a separate syringe 50 is associated with each inflatable member 44 for delivering pressurized fluid thereto. While in this exemplary embodiment the source of pressurized fluid is external to the body, it is to be understood that the pressurized fluid source may comprise a pressurized fluid tank that is internal to the body at the time the inflatable members 44 are inflated.

Alternatively, the inflatable members 44 may be filled with some other substance or structure, such as micro beads that can be pressurized into the members to provide the required rigidity.

It is also within the scope of the invention to utilize magnetorheological fluids to inflate the inflatable members 44. In such a circumstance, the magnetorheological fluid is injected into the inflatable members as a fluid, but becomes a viscoelastic solid upon application of a magnetic field to the fluid just prior to energizing the ablation electrodes. After a viscoelastic solid state is achieved, which creates the desired rigidity of the inflatable members 44, the flexible member 40 is cinched and the ablation electrodes are activated to ablate the tissue in question.

Referring back to FIGS. 2 and 3, the elongated flexible member 40 includes two segments that are connected to one another a resilient link 52, such as a loop, which has comparable resiliency characteristics to the cinching ring 34, so that substantially uniform clamping pressure is applied across the compressed tissue. Also, appropriate selection of the material comprising the link 52 and ring 34 permits some control over the amount of clamping pressure achieved.

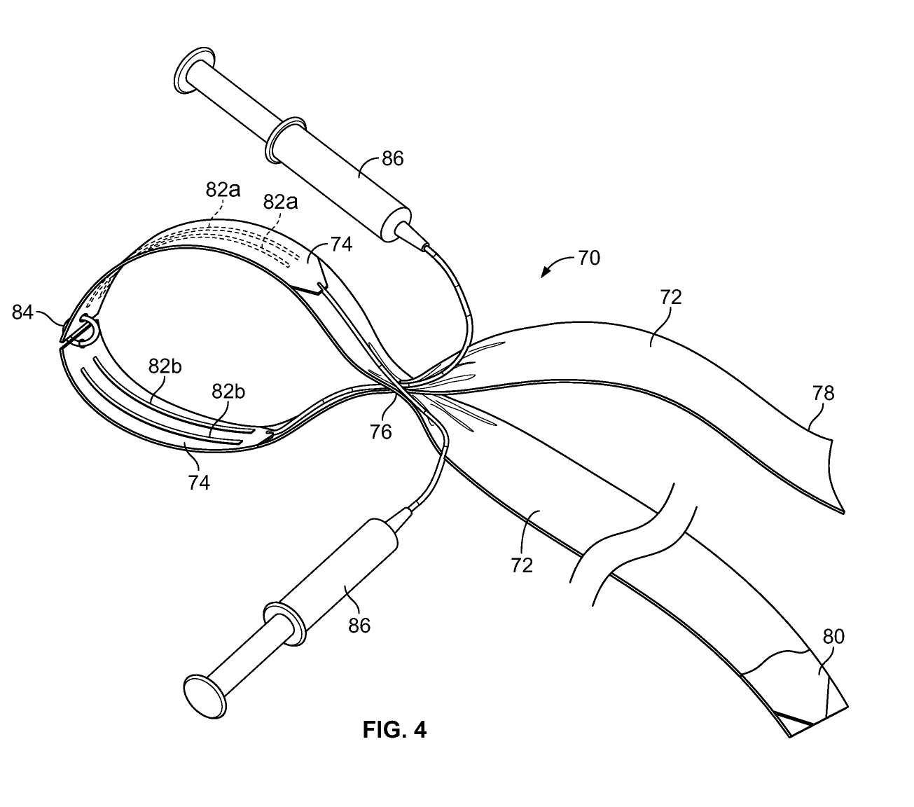

A further alternative, see FIG. 4, similar to the second exemplary embodiment 40, is to provide a flexible ablation clamp 70 comprising an elongated flexible member 72 that supports at least one pair of elongated inflatable members 74 that are spaced apart on opposite portions the elongated flexible member. Each inflatable member 74 may include a semi-rigid elongated member or beam (not shown) associated therewith. In this exemplary embodiment, the flexible member 72 includes an aperture 76 that receives another section of the elongated flexible member (e.g., a “lasso” configuration) in order to cinch opposing parts of the flexible member toward one another when the ends 78, 80 of the flexible member are drawn taut around the tissue to be ablated. As with the second exemplary embodiment 40, this third exemplary embodiment includes a pair of elongated electrodes 82a, 82b associated with corresponding surfaces so what when the flexible member 72 is looped around the tissue to be ablated, the elongated electrodes are oriented to face one another. An RF power source (not shown) is in communication with the electrodes 82a, 82b to provide RF energy. As with the second embodiment, this third embodiment 70 also includes a link 84 coupling the inflatable member 74 and corresponding syringes 86 to inflate the members 74 with pressurized fluid.

As a still further alternative, the ends of the flexible member may be provided with interlocking structures that, when connected, proximate the sleeves about the atrium. One such self-latching structure could be a hook and loop fastener (i.e., Velcro) that is provided on the surfaces of the elongated flexible member.

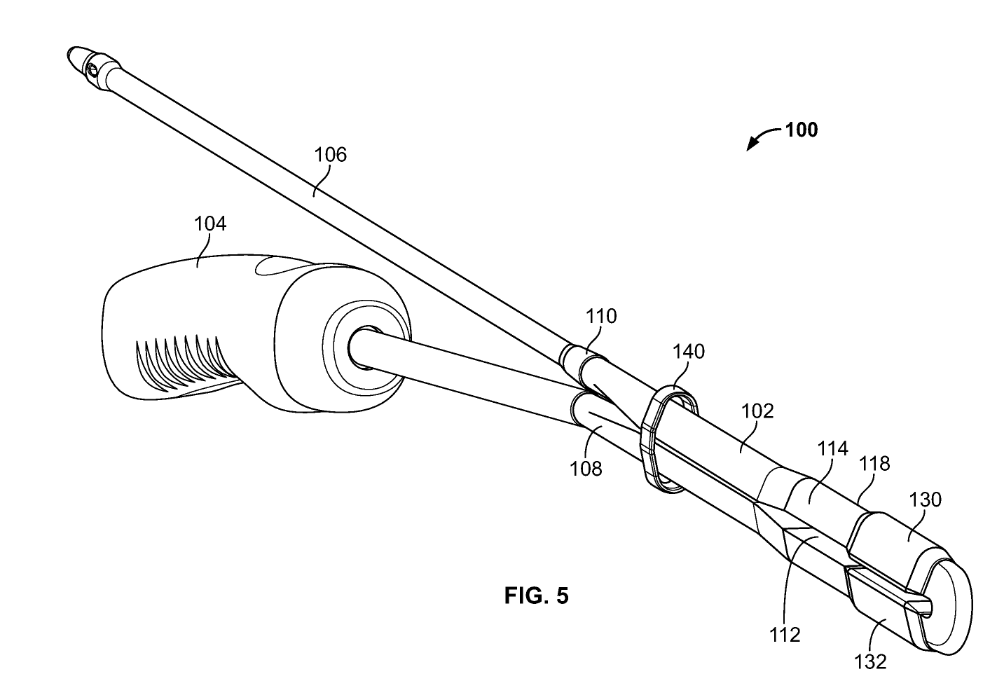

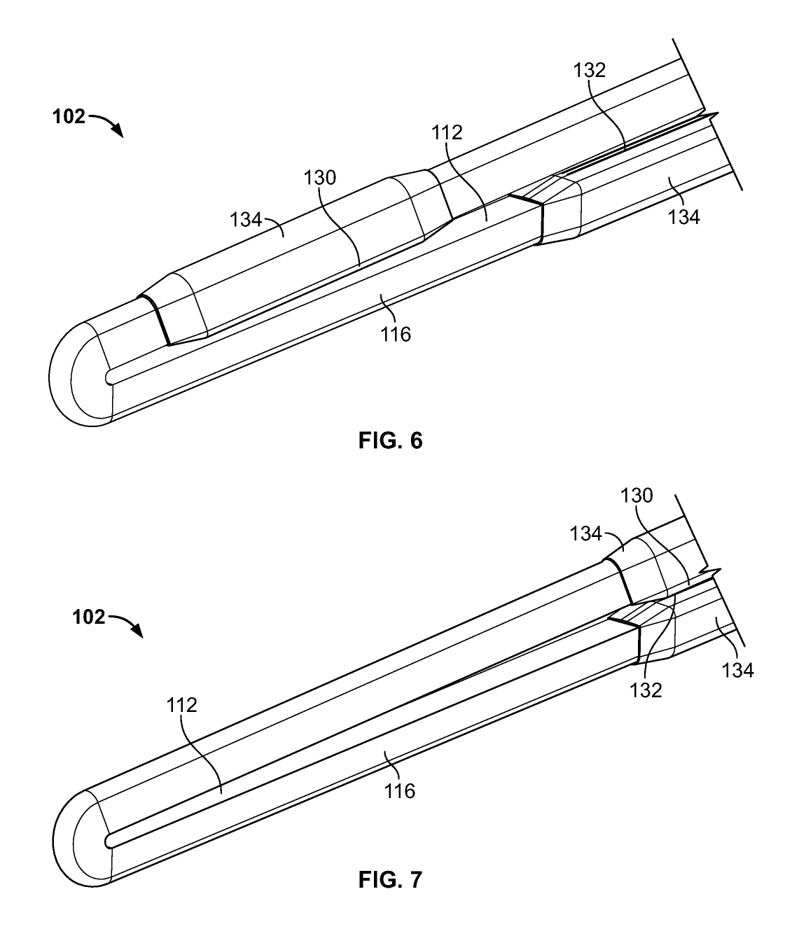

Referring to FIGS. 5-8, another exemplary flexible ablation clamp 100 comprises an elongated flexible member 102 having a length sufficient for it to be introduced into the body of a patient through minimally invasive access orifice (not shown) and encompass predetermined bodily tissue while a handle 104 and a repositioning device 106 are mounted to the ends 108, 110 of the flexible member 102.

The elongated flexible member 102 includes a rounded triangular cross-section with a generally planar interior surface 112 that intersects opposing face surfaces 114, 116 that intersect one another at a rounded apex 118. In this manner, the interior surface 112 and the face surfaces 114, 116 generally comprise the three sides of the rounded triangular cross-section. However, other geometries may also be employed for the elongated flexible member 102, preferably to allow both ends 108, 110 of the elongated flexible member 102 to freely pass through a minimally invasive orifice. While the dimensions may vary substantially, an exemplary length of the elongated flexible member 102 comprises 24 to 36 inches.

In this exemplary embodiment, the flexible member 102 comprises an elastomeric material such as, without limitation, Pellethane polyurethane, Series 2363, available from The Dow Chemical Company (www.dow.com). As will all elastomeric material, the material exhibits an elasticity that approximates a spring rate.

The flexible member 102 may include pockets (not shown) formed on or adjacent one or both of its ends 108, 112 (see, e.g., pocket 18 in FIGS. 2 and 3) that are adapted to cooperate with a positioning tool or instrument, such as the LumiTip dissector available from AtriCure, Inc., West Chester, Ohio (www.atricure.com), for guiding the flexible member around the pulmonary veins.

At least two electrodes 130, 132 are mounted to the flexible member 102. The electrodes 130, 132, in exemplary form, may be sized in length from between about 10 mm and 70 mm, depending upon the intended application of the flexible ablation clamp 100. Each electrode 130, 132 is in electrical communication with a power source (not shown) in order to power on and off the electrodes as desired. Each electrode 130, 132 may be incorporated into a sleeve 134 that fits over the flexible member 102 and may or may not be repositionable along the length of the flexible member. Alternatively, each electrode 130, 132 may be integrated into the flexible member 102 and have a static position. A further alternative provides for the flexible member 102 having a longitudinal cavity along which the electrodes 130, 132 may be positioned and repositioned along the length of the flexible member. The flexible member 102 individually or in combination with the electrodes 130, 132 (or the electrodes by themselves) is operative to provide a rigid structure whereby uniform force profile is established with respect to the tissue to be ablated.

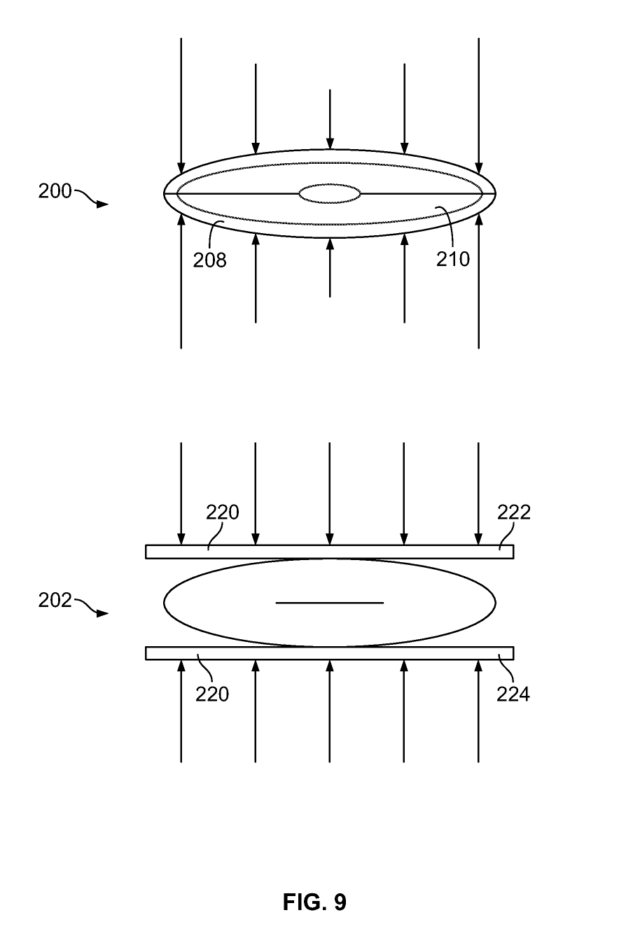

Referring to FIG. 9, an exemplary set of diagrams represents at least some of the advantages of the embodiments of the instant disclosure. While it is known to use flexible ablation devices, these devices are not operative to provide a uniform force profile. An exemplary diagram 200 depicts forces applied by a flexible ablation device 208 and reflects that the flexible nature of the ablation device, while helpful to position the device prior to ablation, is a hindrance to a complete ablation across a biological conduit 210, such as a vein. Specifically, diagram 200 shows that the ends of the biological conduit receive significantly more downward (i.e., pushing) force from the flexible ablation device in comparison to the middle of the conduit. In contrast, diagram 202 depicts threes applied by a rigid ablation device 220 comprising at least two independent segments 222, 224. The rigid nature of the independent segments 222, 224 may retard expedient positioning of the device and because the ends are free, the force profile commonly decreases as the distance from the free ends decreases. In contrast to the foregoing, diagram 204 depicts the uniform force profile exhibited when correctly using the embodiments of the instant disclosure.

The flexible nature of the exemplary embodiments of the instant disclosure allows for expedient positioning of the ablation device, while the rigid nature of a portion of the device during ablation is operative to apply a uniform force profile across the tissue in question (e.g., a biologic conduit). Specifically, the exemplary embodiments include constraints proximate the ends of the rigid portions that are operative to provide a uniform force profile. As has been discussed previously and will be discussed also below, the constraints at the ends of the rigid portions may be rigid themselves or function as a spring and exhibit a spring rate.

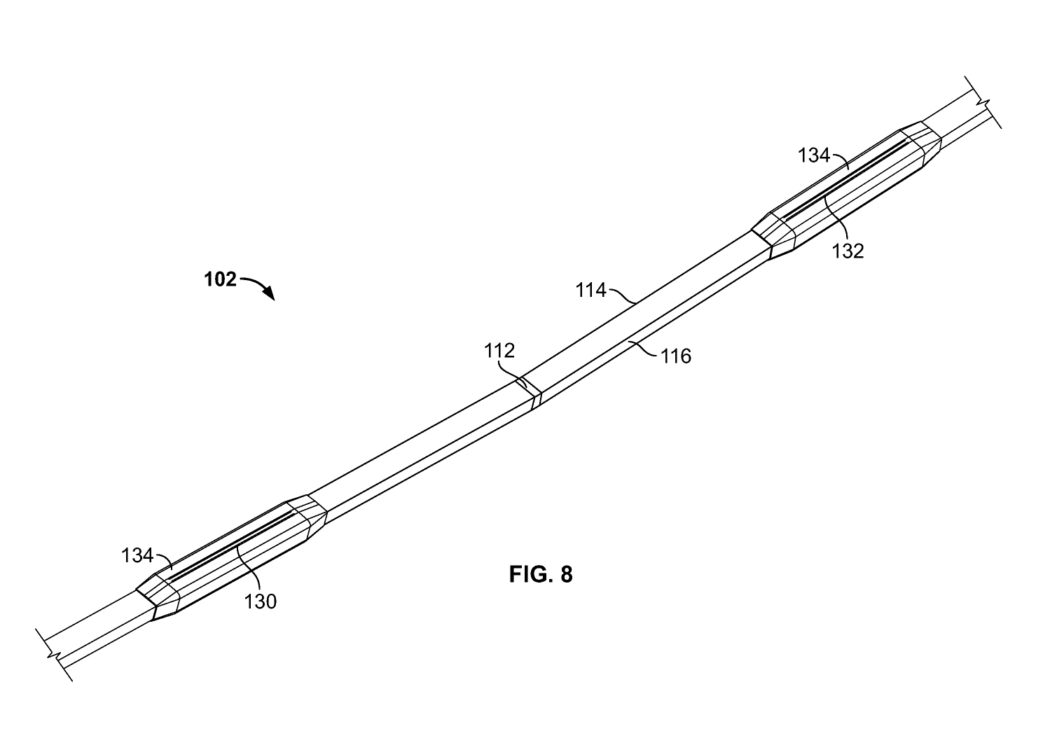

Referring back to FIGS. 5-8, the exemplary flexible ablation clamp 100 also a cinching ring 140 that is repositionable along the length of the flexible member 102 and adapted to circumscribe a doubled over portion of the flexible member. In this exemplary embodiment, the flexible member 102 includes an inherent spring rate that would orient the flexible member 102 is a substantially linear fashion as shown in FIG. 8, but for a compressive force orienting the flexible member as shown in FIGS. 5-7. In exemplary form, the cinching ring 140 has substantially the same spring rate as that of the flexible member when doubled over on itself in order to clamp tissue between the electrodes 130, 132 and establish a uniform force profile. This uniform three profile also operates to completely seal off a tissue conduit extending perpendicularly between the electrodes 130, 132 when the cinching ring 140 is moved away from the electrodes. After ablation, the cinching ring 140 may be severed to allow easier withdrawal of the flexible member from the body.

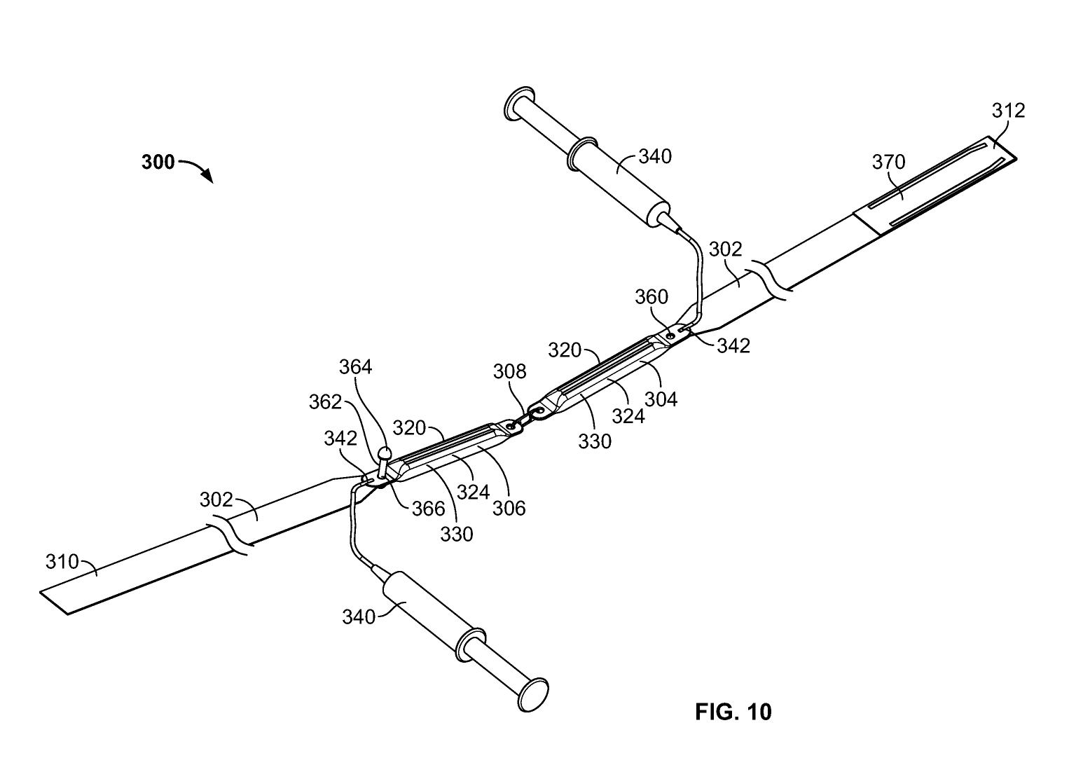

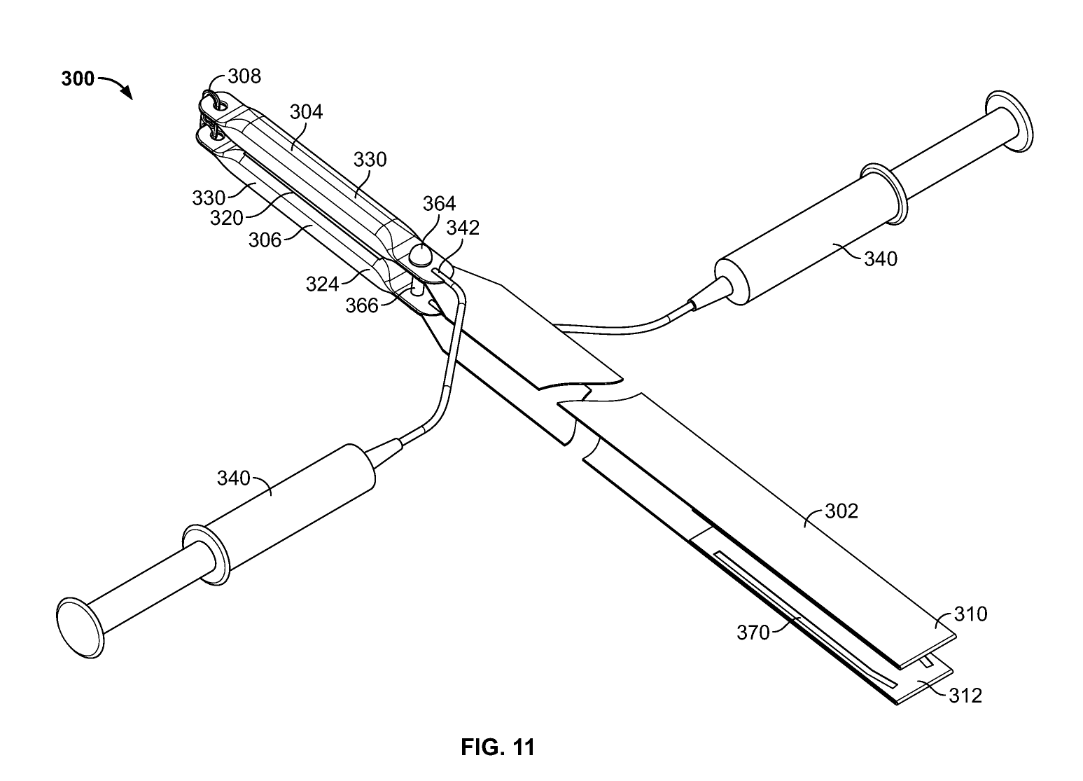

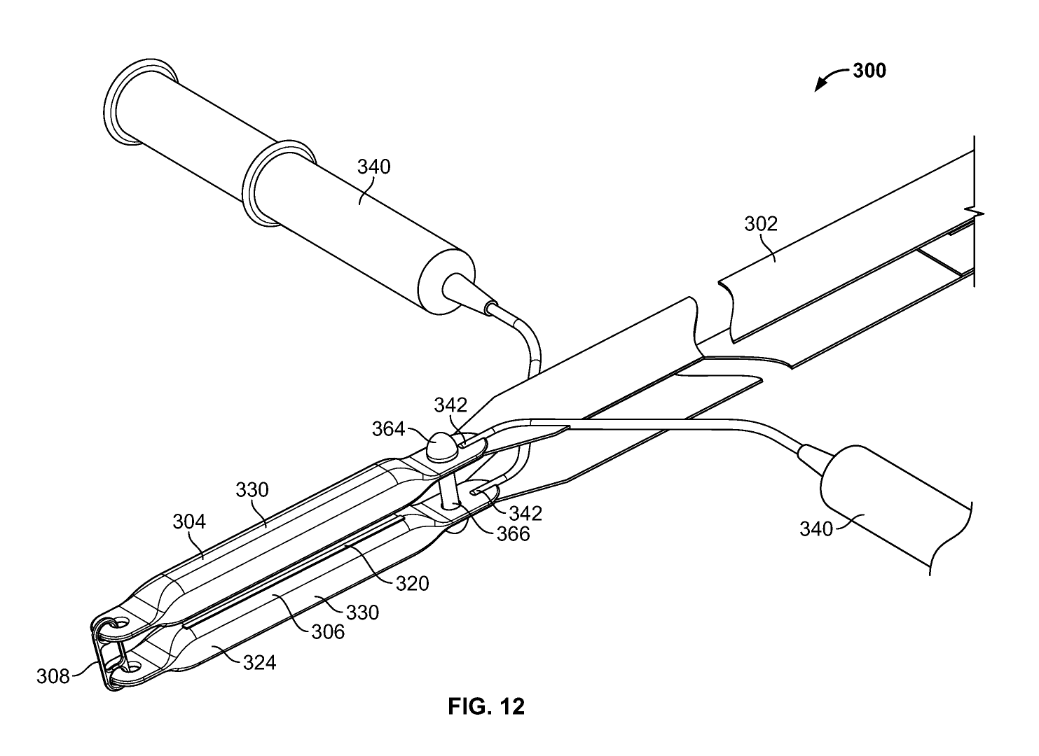

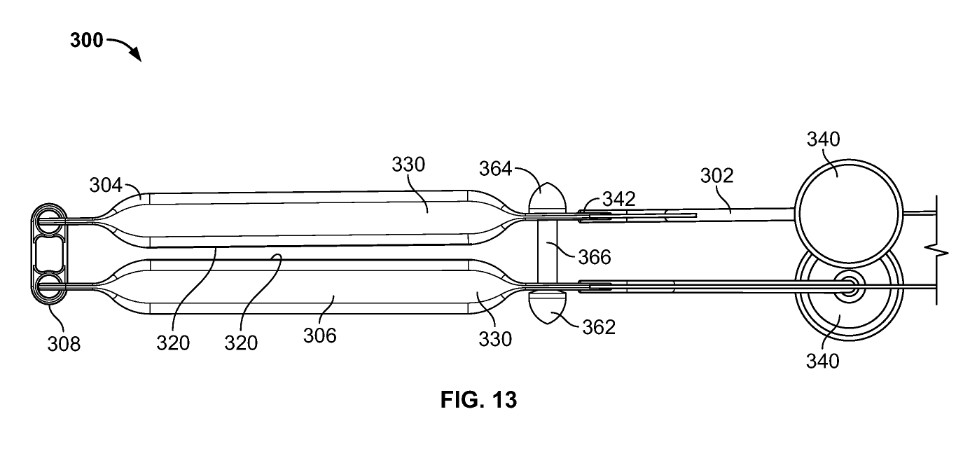

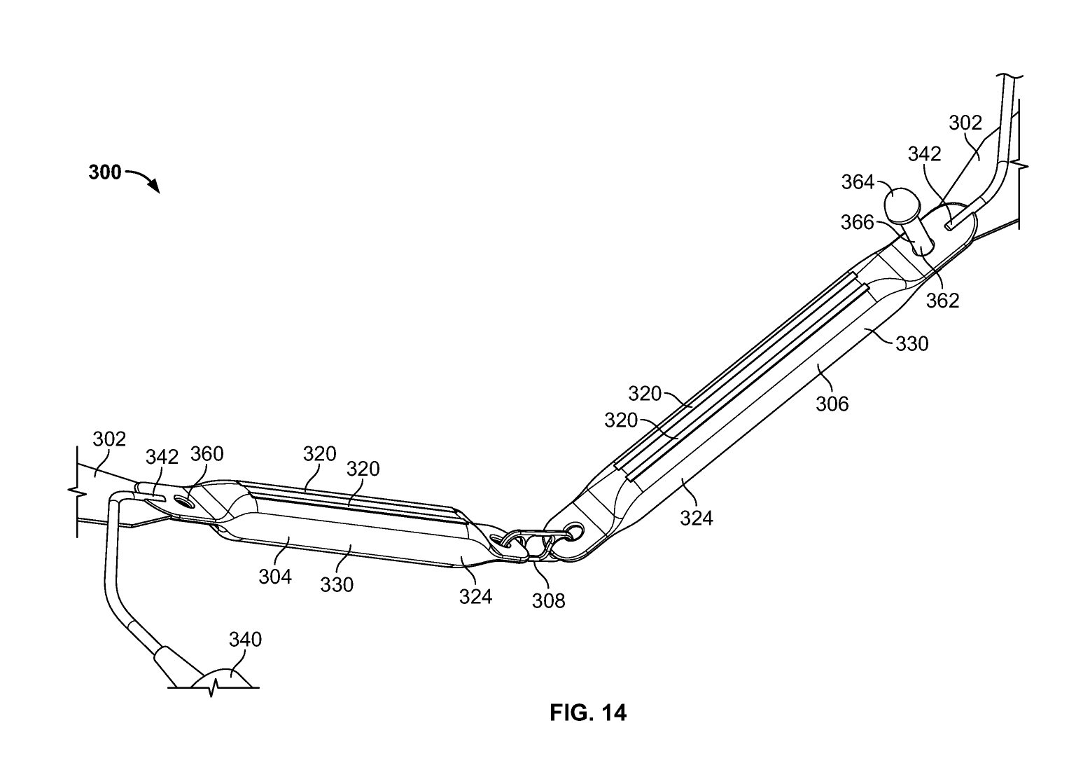

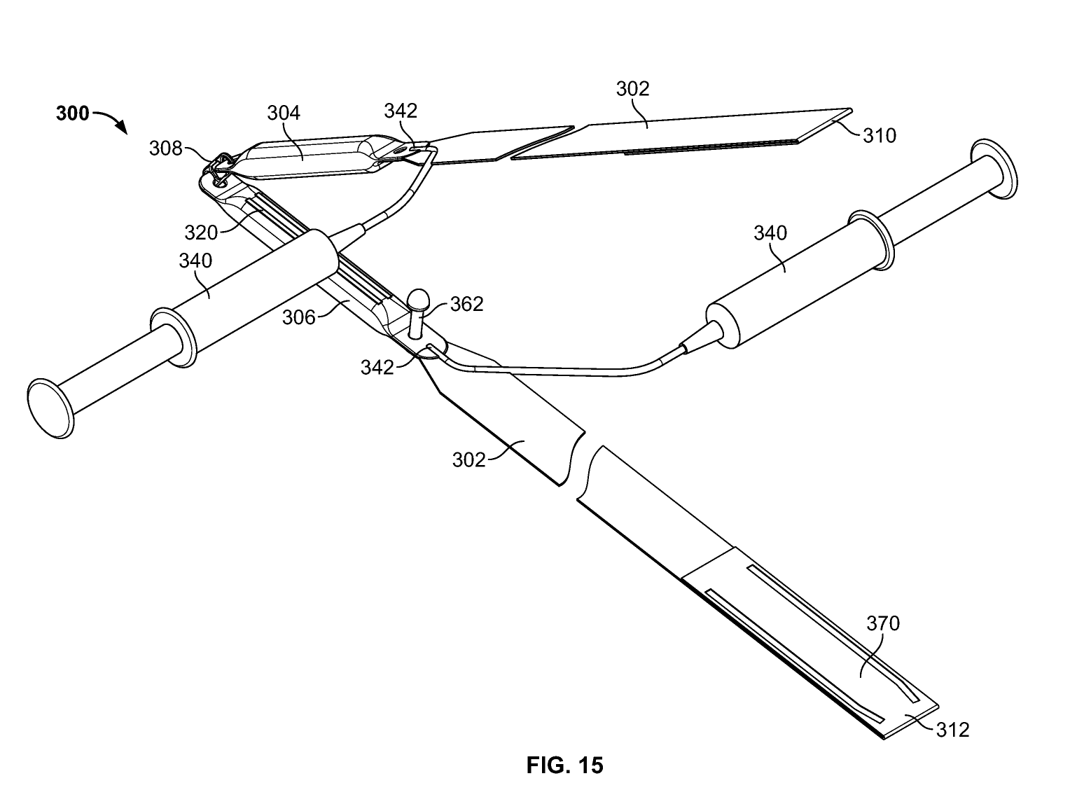

Referencing FIGS. 10-15, a further exemplary embodiment of a flexible ablation clamp 300 comprises an elongated flexible member 302 having a first ablation section 304 and a second ablation section 306 coupled to one another by an elastic link 308. Respective ends 310, 312 of the flexible member 302 are adapted to be coupled to a guide wire (not shown).

Each ablation section 304, 306 includes at least one ablation electrode 320 this is operatively coupled to a power source such as, without limitation, radio frequency and microwave. In this exemplary embodiment, each ablation section includes a pair of linear ablation electrodes 320 that have the same length, are oriented in parallel to one another, and are spaced apart. As will be described in more detail, hereafter, the ablation electrodes 320 are positioned along a top surface 324 of the ablation sections 304, 306, which eventually becomes the interior surface when the sections overlie one another for an ablation procedure.

Each ablation section 304, 306 also includes an inflatable bladder 330 that is operatively coupled to a pressurized fluid source 340 that delivers fluid at sufficient pressure in order to render each ablation section rigid just prior to and during the ablation procedure. By way of example, exemplary fluids include, without limitation, air, cryogenic fluids, saline solutions, and water. Exemplary pressurized fluid sources include, without limitation, syringes, fluid pumps, compressors, and pressurized fluid tanks. In exemplary form, each inflatable bladder 330 includes a coupling 342 for establishing fluid communication between the bladder and a respective syringe 340 to deliver a pressurized fluid.

The first ablation section 304 includes a scaled orifice 360 located on the opposite end permanently coupled to the second ablation section 306. This sealed orifice 360 is not in fluid communication with the interior of the bladder 330, but is adapted to receive a retainer 362 associated with the second ablation section 306 for selectively coupling and decoupling corresponding ends of the ablation sections to one another. In this exemplary embodiment, the retainer 362 comprises a resilient stud having a tapered head 364 extending from a linear shaft 366. The base of the shaft 366 is coupled to the end of the second ablation section 306 opposite the link 308.

The flexible member 302 may include pockets 370 formed on or adjacent one or both of its ends 310, 312 that are adapted to cooperate with a positioning tool or instrument, such as the LumiTip dissector available from AtriCure, Inc., West Chester, Ohio (www.atricure.com), for guiding the flexible member around the pulmonary veins.

In operation, a free end of a guide wire is initially routed around the tissue to be ablated. After the surgeon has routed the guide wire in a looping arrangement around the tissue to be ablated, the free end is pulled so that the flexible member 302 follows the path of the guide wire and the ablation sections 304, 306 loop around the tissue to be ablated. At this point, the first ablation section 304 is folded over the second ablation section and the ends opposite the link 308 are coupled to one another. This coupling is accomplished by inserting the tapered head 364 of the retainer 362 into and through the scaled orifice 360. Because the largest diameter portion of the tapered head 364 is larger than the diameter of the sealed orifice 360, once the tapered head passes beyond the sealed orifice, the tapered head is operative to couple the ablation sections 304, 306 opposite the link 308. Because the inflation of the bladders 330 is used to make the sections 304 rigid, as opposed to creating form fit around the tissue in question, the point in time at which the bladders 330 are inflated is arbitrary with respect to when the tapered head 364 is inserted into and through the sealed orifice 360.

After the bladders 330 are inflated and the ablation sections 304, 306 are folded over one another with the ends coupled together, the ablation electrodes 320 may be activated to create an ablation lesion across the tissue clamped between the sections. The ablation lesion is created while the bladders 330 provide a substantially uniform pressure profile across the tissue being ablated. Again, as discussed above, the bladders 330 are not inflated to contour to the tissue being ablated; but, rather, the bladders are inflated to impart sufficient rigidity in order to apply a substantially uniform pressure profile to the tissue being ablated.

Subsequent to ablation, the retainer 362 may be disengaged from the sealed orifice 360 so that the sections 304, 306 no longer are folded over one another. At generally the same time, the bladders 330 may be partially or completely deflated to allow easier removal of the flexible ablation clamp 300 from the body.

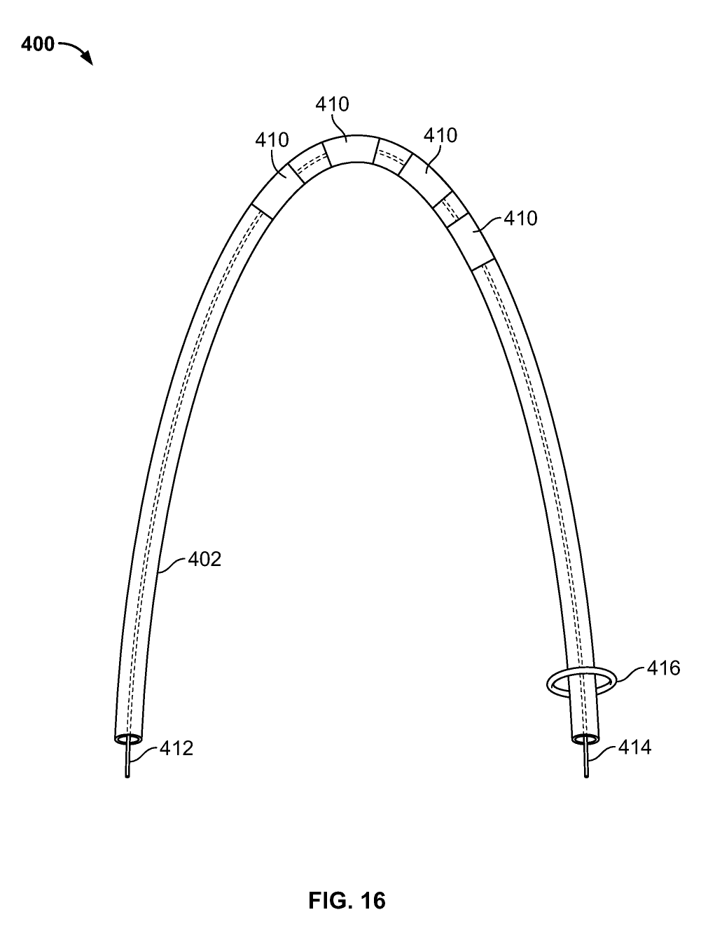

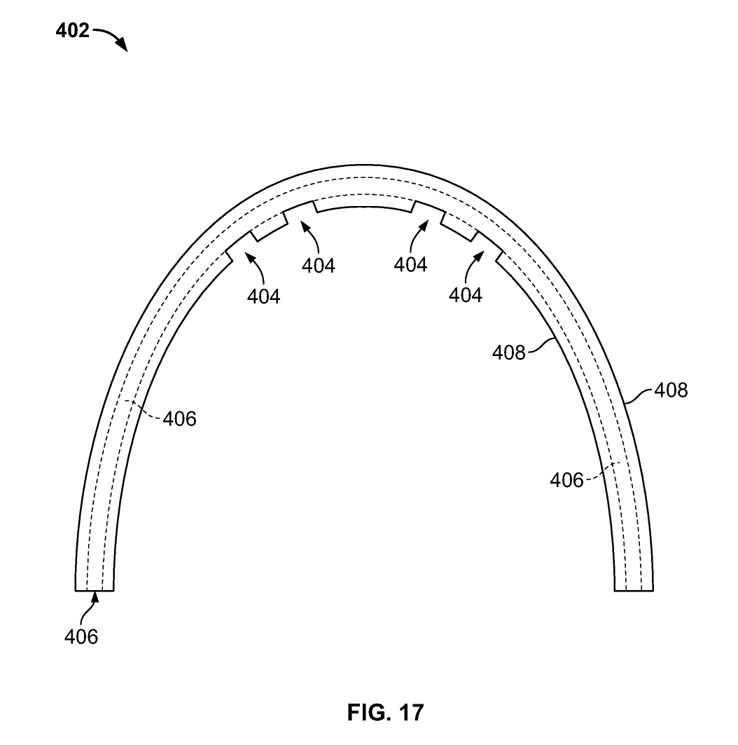

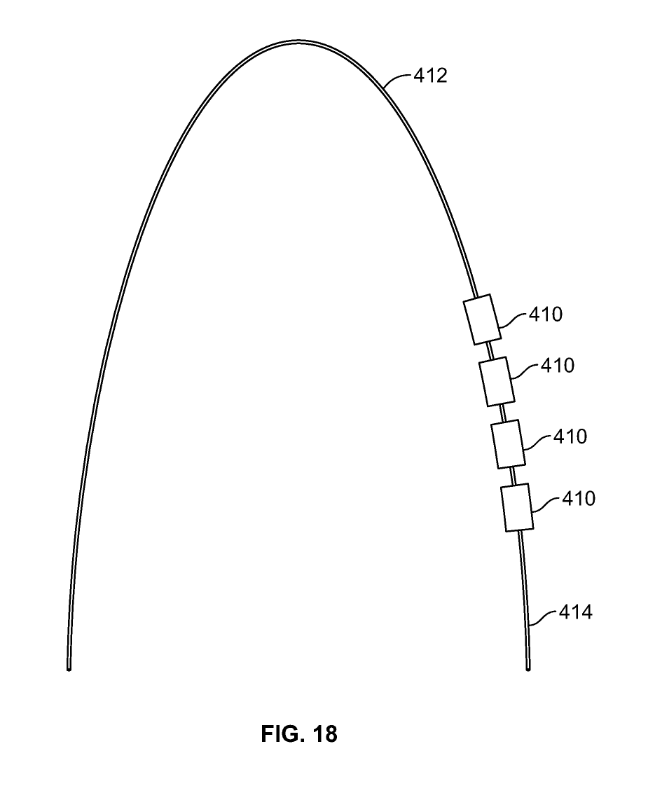

Referring to FIGS. 16-18, yet a further exemplary embodiment of a flexible ablation clamp 400 comprises a hollow elongated flexible member 402 having plurality of windows 404 that extend from the hollow interior portion 406 to an exterior 408 of the flexible member. The hollow elongated flexible member 402 may be formed, at least in part, of an elastomer, such as a polyethylene or polyurethane. One such material suitable for this use is Pellethane polyurethane, Series 2363, available from The Dow Chemical Company (www.dow.com).

The hollow interior portion 406 is sized to allow a plurality of ablation electrodes 410 to be longitudinally repositioned along the length of the flexible member 402. In this exemplary embodiment, a pair of ablation electrodes 410 may include an associated guide filament 412 that extends within the hollow interior portion 406. Alternately, the electrodes 410 may be repositioned without the use of a guide filament 412. The guide filament 410 has a substantially narrower diameter than that of the hollow interior portion 406. Exemplary guide filaments 410 include, without limitation, nylon, polyvinylidene fluoride (PVDF, and called fluorocarbon), polyethylene, Dacron and Dyneema (UHMWPE). As will be discussed in more detail below, the guide filament 412 allows the electrodes 410 to be longitudinally repositioned along the length of the hollow interior portion 406 until reaching the desired window 404. When reaching the desired window, the electrodes 410 have direct access to ablate tissue coming in contact therewith.

When the flexible member 402 is initially positioned around the tissue to be ablated, the ablation electrodes may not be located within the hollow interior portion 406. As a result of the electrodes not being within the flexible member 402, the member is more flexible and may more easily be “snaked” around the tissue to be ablated so that portions of the flexible member 402 and associated windows 404 overlie one another (i.e., are folded over one another). Nevertheless, the guide filament 412 is routed through the hollow interior portion 406 so that a free end of the filament may be pulled along the length of the elongated flexible member 402, while an opposing end of the filament is coupled to one or more electrodes 410. After positioning the flexible member 402 so that the windows overlie the tissue to be ablated, the guide filament 412 is pulled along the length of the hollow interior portion 406 to pull the electrodes into position. Specifically, the electrodes 410 are pulled along the hollow interior portion 406 until reaching the desired window 404 so as to allow direct communication between the electrodes and the tissue to be ablated.

The back side of the electrodes, opposite the guide filament 412, includes a transmission line 414 operative to deliver power to the electrodes. In this exemplary embodiment, the electrodes are powered by RF energy from an RF source (not shown). However, other energy sources beyond RF electrodes may be utilized with this exemplary embodiment and the other exemplary embodiments including, without limitation, cryothermia, microwave, laser, intense ultrasound. Moreover, it should be understood that the energy sources may be delivered from either both contact surfaces or only from one side. Though not necessary, it is within the scope of the invention for the electrodes to having lengths substantially longer than that of the windows. Likewise, it is further within the scope of the invention to utilize the electrodes to create sufficient rigidity along part of the length of the flexible member 402 in order to provide a substantially uniform force profile across the tissue to be ablated.

After the electrodes 410 have been positioned with respect to the windows 404, a cinch ring 416 or some other form of catch is positioned proximate the tissue to be ablated. In this exemplary embodiment, the cinch ring or catch 416 is elastic and has a spring rate similar to that of the flexible member 402. In this manner, the resulting structure provides a pair of substantially rigid sections that are coupled together at the ends by elastic links (the flexible member itself 402 at one end of the sections, and the cinch ring or catch 414 at the other end of the sections). At this point, ablation energy may be applied to the electrodes in order to form an ablation lesion across the tissue to be ablated. Subsequent to ablation, the flexible member 402 is removed from the body. This removal may be prefaced by removal of the electrodes 410 or not.

In this exemplary embodiment, the flexible member 402 and electrodes 410 may be disposable so that severing the flexible member subsequent to ablation may be utilized to remove the flexible member from the body.

Any of the foregoing embodiments may include transparent or translucent materials that comprise the flexible members.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. An ablation instrument comprising:
an elastic elongated loop sized in length to at least partially encircle an internal anatomical structure;
a first sleeve and a second sleeve operatively coupled to the elongated loop and repositionable along a length of the elongated loop, the first sleeve and second sleeve each including a firm portion, wherein the firm portion of the first sleeve and the firm portion of the second sleeve each provide a substantially uniform force profile;
an ablation device operatively coupled to the first sleeve and the second sleeve.

2. The ablation instrument of claim 1, wherein the flexible elongated loop includes a pocket that interfaces with a dissector.

3. The ablation instrument of claim 1, wherein the flexible elongated loop is at least one of transparent and translucent.

4. The ablation instrument of claim 1, wherein the flexible elongated loop is elastic.

5. The ablation instrument of claim 1, further comprising a constrictor repositionable along the elongated loop, the constrictor operative to bias the first sleeve toward the second sleeve.

6. The ablation instrument of claim 5, wherein the constrictor comprises a tube received over a portion of the elongated loop.

7. The ablation instrument of claim 5, wherein the constrictor comprises a ring received over a portion of the elongated loop.

8. The ablation instrument of claim 1, wherein the ablation device includes a radio frequency electrode.

9. The ablation instrument of claim 1, wherein:
the ablation device includes a plurality of radio frequency electrodes;
the first sleeve includes at least one of the plurality of radio frequency electrodes;
the second sleeve includes at least one of the plurality of radio frequency electrodes; and
the at least one radio frequency electrode of the first sleeve is of an opposite polarity to the at least one radio frequency electrode of the second sleeve.

10. The ablation instrument of claim 1, wherein the elongated loop includes an orifice sized to allow throughput of a free end of the elongated loop.

11. An ablation instrument comprising:
a discontinuous elongated loop sized in length to be capable of encircling an internal anatomical structure inside a body, the discontinuous elongated loop including a first section joined to a second section by an elastic coupling;
a first inflatable bladder operatively coupled to first section, the first inflatable sleeve including a fluid passageway delivering pressurized fluid thereto, the first inflatable sleeve being generally noncompliant when inflated;
a second inflatable bladder operatively coupled to second section, the second inflatable sleeve including a fluid passageway delivering pressurized fluid thereto, the second inflatable sleeve being generally noncompliant when inflated; and
an ablation device operatively coupled to the first and second inflatable bladders.

12. The ablation instrument of claim 11, further comprising a constrictor repositionable along the discontinuous elongated loop, the constrictor operative to bias the first inflatable bladder toward the second inflatable bladder.

13. The ablation instrument of claim 12, wherein the constrictor comprises a tube received over a portion of the discontinuous elongated loop.

14. The ablation instrument of claim 12, wherein the constrictor comprises a ring received over a portion of the discontinuous elongated loop.

15. The ablation instrument of claim 11, wherein the ablation device includes radio frequency electrodes.

16. The ablation instrument of claim 11, wherein:
the ablation device includes a plurality of radio frequency electrodes;
the first inflatable bladder includes at least one of the plurality of radio frequency electrodes;
the second inflatable bladder includes at least one of the plurality of radio frequency electrodes; and
the at least one radio frequency electrode of the first inflatable bladder is of an opposite polarity to the at least one radio frequency electrode of the second inflatable bladder.

17. The ablation instrument of claim 11, wherein the discontinuous elongated loop is adapted to receive a guide tool.

18. The ablation instrument of claim 17, wherein the discontinuous elongated loop includes a pocket for receiving the guide tool.

19. The ablation instrument of claim 11, wherein the first section and the second section cooperate to form a second elastic coupling, opposite the elastic coupling.

20. The ablation instrument of claim 19, wherein the second elastic coupling includes a shaft with a tapered head that is received within a corresponding opening, wherein the first section includes the corresponding opening and the second section includes the shaft and tapered head.

21. The ablation instrument of claim 11, at least one of the first section and the second section includes an orifice sized to allow throughput of a free end of one of the sections.

22. An ablation instrument comprising:
an elastic ligature loop having an internal longitudinal hollow cavity and at least one window extending from an exterior of the ligature loop into the longitudinal hollow cavity;
a plurality of electrodes being sized to be received within the internal longitudinal hollow cavity and repositionable along a length of the elastic ligature loop, the plurality of electrodes being sized to provide a substantially uniform force profile.

23. The ablation instrument of claim 22, further comprising a filament coupled to the plurality of electrodes.

24. The ablation instrument of claim 23, wherein the cross-sectional area of the filament is substantially less than the cross-sectional area of the internal longitudinal hollow cavity.

25. The ablation instrument of claim 22, wherein the filament comprises at least one of nylon, polyvinylidene fluoride, polyethylene, Dacron, and Dyneema.

26. A method of ablating tissue comprising:
directing a flexible device to at least partially loop tissue to be ablated, the flexible device including at least two ablation electrodes interposed by an elastic coupling;
establishing a substantially uniform force profile across the tissue to be ablated by imparting rigidity to the flexible device proximate the at least two ablation electrodes; and
ablating the tissue.

27. A method of ablating tissue comprising:
directing a flexible device to at least partially loop tissue to be ablated;
repositioning at least two substantially rigid sleeves along the flexible device and into contact with the tissue to be ablated, where the at least two substantially rigid sleeves each include an ablation electrode operative to establish a substantially uniform force profile across the tissue to be ablated, and wherein the at least two substantially rigid sleeves are interposed by an elastic device; and
ablating the tissue.

28. A method of ablating tissue comprising:
directing a flexible device to at least partially loop tissue to be ablated so that at least two inflatable bladders associated with the flexible device are on opposite sides of tissue to be ablated, each of the at least two flexible bladders including an ablation electrode;
inflating the inflatable bladders to impart rigidity to the bladders and establish a substantially uniform force profile across the tissue to be ablated; and
ablating the tissue.
